(19) United States
(12) Patent Application Publication
(10) Pub. No.: US 2022/0280079 A1
(43) Pub. Date: Sep. 8, 2022

(54) SENSOR COMPOSITION AND SENSOR PATCH

(71) Applicant: The Curators of the University of Missouri, Columbia, MO (US)

(72) Inventors: Chang-Soo Kim, Rolla, MO (US); William Stoecker, Rolla, MO (US); Paul Nam, Rolla, MO (US); Mousumi Bose, Rolla, MO (US); Jason Hagerty, Rolla, MO (US)

(73) Assignee: The Curators of the University of Missouri, Columbia, MO (US)

(21) Appl. No.: 17/685,609

(22) Filed: Mar. 3, 2022

Related U.S. Application Data
(60) Provisional application No. 63/156,157, filed on Mar. 3, 2021.

(51) Int. Cl. A61B 5/1455; C09B 67/46; A61B 5/00; C09B 67/02; C09B 47/04

(52) U.S. Cl. CPC A61B 5/14552; C09B 67/009; A61B 5/6833; C09B 67/0097; C09B 47/04; A61B 5/14556; A61B 2562/046

(57) ABSTRACT

Improved sensor compositions comprising a dye are provided. The compositions are generally characterized by at least partially immobilizing the dye on polymeric microparticles and dispersing in a dispersion medium. The dye may comprise an oxygen, humidity, carbon dioxide, temperature, or pH-responsive dye. Also provided is a sensor patch comprising a substrate and the sensor composition comprising a dye.

### STATEMENT REGARDING FEDERALLY SPONSORED RESEARCH OR DEVELOPMENT

This invention was made with Government support under R41NR018126-01 awarded by the National Institutes of Health. The Government has certain rights in the invention.

### FIELD OF THE INVENTION

The present invention provides an oxygen sensor patch comprising polymeric microparticles and a dye, and related methods and compositions.

### BACKGROUND OF THE INVENTION

Oxygen concentration is one the most crucial factors in medical diagnoses, industrial processes, and environmental surveillance. For example, monitoring tissue for oxygen concentration may be undertaken in order to prevent ulcers caused by insufficient blood flow over a prolonged period. Insufficient blood flow and/or ulcers may lead to ischemia and necrosis. Typically, multi-step wound healing processes require adequate oxygen supply at each step. Since oxygen cannot be stored in the cells, a constant supply of oxygen to the cells is necessary for wound healing as well as for the prevention of certain conditions such as ulcers. Therefore, real-time monitoring of oxygen concentration is desirable in the course of medical diagnoses, treatments, etc. Real-time oxygen monitoring is also desirable where oxygen may be deleterious to an item such as a food product or other item for which oxygenation adversely affects the material. For example, it may be desirable to monitor the oxygen in food packaging.

The Clark electrochemistry-based method is typically used in order to measure the partial pressure of oxygen (p_{O2}). This method incorporates the reduction of oxygen at a platinum cathode under an applied potential, typically 0.7 V. The current generated during this process is directly related to the p_{O2}. The major limitations of this method, however, is that it consumes oxygen, which can ultimately alter the actual oxygen concentration. This method also suffers from the possibility of errors due to interference with the magnetic field and/or other deposited elements on the electrode, and is generally only suitable for measurements in a relatively small area. Other available non-invasive, 2D imaging techniques based on radioisotopes (e.g. positron emission tomography) and magnetic resonance (e.g. magnetic resonance oximetry) are expensive and are thus limited to certain specific medical applications.

Therefore, a need exists for an oxygen sensor that is capable of monitoring the oxygen concentration of a particular material or tissue, for example monitoring the transcutaneous oxygen tension of a surface (e.g., a biological tissue), in a cost effective manner. For example, an oxygen sensor in the form of a patch.

A need also exists for sensors that are capable of monitoring the humidity, carbon dioxide, temperature, or pH of a surface (e.g., a biological tissue).

There is also a need for certain sensor compositions that may be used in the preparation of such oxygen, humidity, carbon dioxide, temperature, or pH sensors and have properties that allow for the effective and economical monitoring of said conditions of a particular surface (e.g., biological tissue).

### BRIEF SUMMARY OF THE INVENTION

Among the various aspects of the present invention is the provision of a sensor composition comprising polymeric microparticles; a dye comprising a luminophore that is at least partially immobilized on the polymeric microparticles; and a dispersion medium comprising the polymeric microparticles dispersed therein. Another aspect of the present invention is a sensor patch comprising a substrate and the sensor composition disposed thereon.

Briefly, therefore, the present invention is directed to a sensor composition. The sensor composition comprises polymeric microparticles; a dye comprising a luminophore that is at least partially immobilized on the polymeric microparticles; and a dispersion medium comprising the polymeric microparticles dispersed therein.

The present invention is also directed to a process for preparing a sensor composition. The process comprises mixing polymeric microparticles, a dye comprising a luminophore, and a solvent to form a mixture comprising dye-loaded polymeric microparticles comprising the dye at least partially immobilized thereon. The dye-loaded polymeric microparticles are separated from at least a portion of the solvent in the mixture. The dye-loaded polymeric microparticles are then dispersed in a dispersion medium to form the sensor composition.

The present invention is further directed to a sensor patch. The sensor patch comprises a substrate and a sensor composition disposed thereof, wherein the sensor composition comprises polymeric microparticles; a dye comprising a luminophore that is at least partially immobilized on the polymeric microparticles; and a dispersion medium comprising the polymeric microparticles dispersed therein.

The present invention is also directed to a sensor patch comprising a substrate and a sensor composition disposed on the substrate, wherein the sensor composition comprises dye-loaded polymeric microparticles comprising a dye at least partially immobilized on the polymeric microparticles.

The present invention is further directed to methods of preparing a sensor patch comprising applying a sensor composition, wherein the sensor composition comprises polymeric microparticles; a dye comprising a luminophore that is at least partially immobilized on the polymeric microparticles; and a dispersion medium comprising the polymeric microparticles dispersed therein, to a substrate and drying the substrate to at least partially remove the dispersion medium.

The present invention is also directed to methods of using the sensor patch of the invention. For example, a method of monitoring a biological tissue. The method comprises applying the sensor patch of the present invention to the biological tissue and analyzing the luminescence of the sensor patch. An alternative method comprises applying the sensor patch of the present invention to a biological tissue comprising a wound dressing covering at least a portion of the surface of the biological tissue and analyzing the luminescence of the sensor patch.

The present invention is also directed to methods of using the sensor patch of the present invention to monitor the oxygen, humidity, carbon dioxide, temperature, or pH of a sample. For example, monitoring the oxygen, humidity, carbon dioxide, temperature, or pH of a sample that is oxygen-sensitive or perishable, such as food, medical supplies, or other items.

Other objects and features will be in part apparent and in part pointed out hereinafter.

### BRIEF DESCRIPTION OF THE DRAWINGS

FIG. 1A is an exemplary configuration of a sensor patch comprising an array of sensor dots.

FIG. 1B is an exemplary configuration of a sensor patch comprising an array of sensor dots and an underlying wound dressing layer between at least a portion of the surface of the sample and the sensor patch.

FIG. 1C is an example of a sensor patch applied to the surface of a biological tissue, further comprising a reference electrode.

FIG. 1D is a prototype printing device.

FIG. 2A is a schematic of oxygen sensor patch attached with calibration chamber.

FIG. 2B is a smart phone imaging configuration.

FIG. 3A is the luminescence response captured by the smart phone and saved in RAW format, exposed to 0% oxygen.

FIG. 3B is the luminescence response captured by the smart phone and saved in RAW format, exposed to 4% oxygen.

FIG. 3C is the luminescence response captured by the smart phone and saved in RAW format, exposed to 12% oxygen.

FIG. 4A shows the variation of microparticle size with PVP-to-styrene-monomer ratio.

FIG. 4B shows the size distribution for three types of polystyrene microparticles.

FIG. 5A shows the absorption spectra of the PtTFPP dye and dye encapsulated in a polystyrene microparticle (PtTFPP/PS) suspended in 50% ethanol.

FIG. 5B shows the emission spectra of the PtTFPP dye and dye encapsulated in a polystyrene microparticle (hereinafter PtTFPP/PS) suspended in 50% ethanol.

FIG. 6A shows the variation of luminescence intensity of sensor patch with polystyrene particle size.

FIG. 6B shows the variation of luminescence intensity of sensor patch with dye concentration.

FIG. 7A shows the phosphorescence intensity emitted by the single sensor dot oxygen sensor patch when excited with 470 nm light at different oxygen concentration.

FIG. 7B is the Stern-Volmer plot for a single sensor dot oxygen sensor patch, excited with 470 nm light.

FIG. 8 is the Stern-Volmer plot for an array of sensors on an oxygen sensor patch, excited with 405 nm light.

FIG. 9 reports the operational stability and response time of a sensor patch when switching alternately between 100% nitrogen and 100% oxygen environment.

### DETAILED DESCRIPTION OF THE INVENTION

The present invention provides a sensor composition comprising a luminophore that is at least partially immobilized on polymeric microparticles. The composition exhibits desirable properties for preparing a non-invasive, rapid, and cost-effective sensor patch for measuring conditions on a surface (e.g., biological tissue). For example, the sensor patch may be useful for measuring one or more of oxygen, humidity, carbon dioxide, temperature, or pH on a surface. The present invention also provides a sensor patch comprising a substrate and a sensor composition and methods of using the sensor patch. The present invention also provides methods for preparing the sensor composition and sensor patch.

In one embodiment, the present invention provides a sensor composition comprising polymeric microparticles; an oxygen-responsive dye comprising a luminophore that is at least partially immobilized on the polymeric microparticles; and a dispersion medium comprising the polymeric microparticles dispersed therein.

In another embodiment, the present invention provides a process for preparing the sensor composition. The process comprises mixing polymeric microparticles, an oxygen-responsive dye comprising a luminophore, and a solvent to form a mixture comprising dye-loaded polymeric microparticles comprising the oxygen-responsive dye at least partially immobilized thereon; separating the dye-loaded polymeric microparticles from at least a portion of the solvent in the mixture; and dispersing the dye-loaded polymeric microparticles in a dispersion medium to form the sensor composition.

Oxygen concentration is one of the most crucial factors in medical diagnoses, industrial processes, and environmental surveillance. For example, in a medical context, tissue oxygen monitoring may be undertaken in order to prevent ulcers caused by insufficient blood flow over a prolonged period. Insufficient blood flow and/or ulcers may lead to ischemia and necrosis. Typically, multi-step wound healing processes require adequate oxygen supply at each step. Since oxygen cannot be stored in the cells, a constant supply of oxygen to the cells is necessary for wound healing as well as for the prevention of certain conditions such as ulcers. Therefore, real-time monitoring of oxygen concentration is desirable in the course of medical diagnoses, treatments, etc.

In certain other industrial and environmental processes and surveillances, it may be desirable to monitor the oxygen concentration of a certain object for purposes of evaluating cell metabolism, food conditions (e.g., for packaged foods), water quality, etc.

Other useful factors in medical diagnoses, industrial processes, and environmental surveillance include, for example, humidity, carbon dioxide concentration, temperature, or pH.

Optical sensing technology has recently gained increased attention due to its potential for numerous applications, a relatively high sensitivity with respect to measurements, and the ability to make such measurements without consuming the subject being measured (e.g., without consuming the oxygen). Optical sensors can be classified based on two operating methodologies: absorption and luminescence.

In the context of oxygen measurement, the absorption method utilizes the contrast between oxygenated and deoxygenated hemoglobin and serves as the basis for pulse oximetry. The absorption method is a useful clinical tool for determining the oxygen level in a blood sample. However, this method is not particularly suited for determining the oxygen level in a biological tissue.

The luminescence (e.g., photoluminescence) detection method is a non-invasive technique that allows for direct quantification of various conditions such as oxygen concentration at a certain photoluminescence-based optical sensor. This method allows for precise points of measurement as well as 2D surface monitoring. In general, luminescence (e.g., photoluminescence) detection is a rapid and real-time process of measuring various conditions (e.g., oxygen concentration, humidity level, carbon dioxide concentration, temperature, or pH level) that allows for relatively sensitive measurements. These attributes make it a desirable technique for a wide variety of applications.

Photoluminescence-based optical sensors typically operate through an energy exchange mechanism. Luminescent molecules are excited to a higher energy state when stimulation with light waves, and emit light of a longer wavelength when they return to the ground energy state. Dynamic collisions of molecular oxygen with excited molecules quench the luminescent emission, resulting in diminished luminescent intensity. Dynamic quenching is completely reversible and follows the Stern-Volmer equation (1), where I₀ and I are intensity of the fluorophore in absence and presence of oxygen respectively, Kₛᵥ is the Stern-Volmer constant, and p_{O2} represents the partial pressure of oxygen.

$$\frac{{I}_{0}}{I} = 1 + {K}_{SV} {p}_{{O}_{2}}$$

Metalloporphyrin-based fluorophores are particularly popular due to their intense red phosphorescence. Metalloporphyrin-based fluorophores also generally exhibit a larger Stokes' shift, longer lifetimes, higher chemical stability, etc. Halogen substitution further generally improves the photostability of the compound. In particular, fluorine substituted Pt(II) porphyrin (PtTFPP) has been used for intensity-based sensing systems.

Similarly, the luminescent molecules (e.g., fluorophores) may be responsive to or excited by, for example, changes in the humidity level, carbon dioxide concentration, temperature, or pH at a given surface.

Monitoring various conditions (e.g., oxygen concentration, humidity level, carbon dioxide concentration, temperature, or pH), either in gas or dissolved in a liquid, requires that the fluorophores be firmly immobilized on a host material. For example, the host material may be a polymeric material. The host polymeric material should be compatible and inert with respect to the fluorophore, provide long-term stability, and have suitable permeability and diffusion rates in order to achieve the desired sensitivity in the measurements. Silicone rubber is a commonly used FDA-approved polymer. However, it has very high oxygen permeability, which results in a narrow linear dynamic range for the measurement of oxygen concentration. Polystyrene is an inert, biocompatible material with oxygen permeability in the physiological range of p_{O2} (0-21% oxygen). Use of polystyrene particles results in a single exponential calibration curve with a single quenching constant. Therefore, polystyrene may be used as host material to monitor cell metabolism and transcutaneous wound healing status, for example, through measuring the oxygen concentration. The polystyrene particles can be synthesized in nano- to micro-particle size.

Sensor Composition

The present invention is directed to a sensor composition comprising a dye. In one embodiment, the sensor composition comprises polymeric microparticles; an oxygen, humidity, carbon dioxide, temperature, or pH-responsive dye that is at least partially immobilized on the polymeric microparticles; and a dispersion medium comprising the polymeric microparticles dispersed therein. In yet another embodiment, the sensor composition comprises polymeric microparticles; an oxygen, humidity, carbon dioxide, temperature, or pH-responsive dye comprising a luminophore that is at least partially immobilized on the polymeric microparticles; and a dispersion medium comprising the polymeric microparticles dispersed therein.

Dissolving the dye in a polymer/solvent mixture is the most common method of immobilizing a dye. In another example, a mixture comprising the polymeric microparticles and oxygen, humidity, carbon dioxide, temperature, or pH-responsive dye may be centrifuged to at least partially immobile the oxygen, humidity, carbon dioxide, temperature, or pH-responsive dye on the polymeric microparticles. In another embodiment, the oxygen, humidity, carbon dioxide, temperature, or pH-responsive dye may be encapsulated in the polymer microparticles to minimize leaching of the fluorophore. In certain embodiments, oxygen, humidity, carbon dioxide, temperature, or pH-responsive dyes can be immobilized on the polymeric microparticles utilizing a surface adsorption technique.

In one embodiment, the oxygen, humidity, carbon dioxide, temperature, or pH-responsive dye comprises a luminophore. In another embodiment, the oxygen, humidity, carbon dioxide, temperature, or pH-responsive dye comprises a luminophore comprising a fluorophore or a phosphor. In one embodiment, the oxygen, humidity, carbon dioxide, temperature, or pH-responsive dye comprises a fluorophore. For example, the oxygen-responsive dye may comprise a fluorophore selected from the group consisting of platinum(II) meso-tetra(pentafluorophenyl)porphin (PtTFPP), platinum(II) meso-tetraphenyl tetrabenzoporphine (PtTPTBP), and combinations thereof.

In certain embodiments, the oxygen, humidity, carbon dioxide, temperature, or pH-responsive dye of the present invention may comprise a luminophore that is selected based on the peak emission wavelength. For example, a luminophore having a peak emission wavelength of from about 300 nm to about 800 nm, from about 300 nm to about 700 nm, from about 300 nm to about 600 nm, from about 300 nm to about 500 nm, from about 310 nm to about 490 nm, from about 320 nm to about 480 nm, from about 330 nm to about 470 nm, from about 340 nm to about 460 nm, from about 350 nm to about 450 nm, from about 360 nm to about 440 nm, from about 370 nm to about 430 nm, from about 380 nm to about 420 nm, or from about 390 nm to about 410 nm.

Although reference is made herein to an oxygen, humidity, carbon dioxide, temperature, or pH-responsive dye comprising a luminophore, it is understood that the present invention is more generally directed to the use of an oxygen, humidity, carbon dioxide, temperature, or pH-responsive dye.

The polymeric microparticles of the present invention may comprise any polymeric material suitable for immobilizing an oxygen, humidity, carbon dioxide, temperature, or pH-responsive dye thereon. In certain embodiments, the polymeric microparticles may be selected such that they are inert to the oxygen, humidity, carbon dioxide, temperature, or pH-responsive dye. In another embodiment, the polymeric microparticles are selected based on the relative oxygen permeability and diffusion rates, for example, in order to achieve the desired sensitivity in oxygen concentration measurements.

For example, the polymeric microparticles may comprise polystyrene. In one embodiment, the polymeric microparticles comprise polystyrene microparticles.

In certain embodiments, the polymeric microparticles have an average particle size of from about 300 nm to about 1200 nm, from about 400 nm to about 1200 nm, from about 500 nm to about 1200 nm, from about 600 nm to about 1200 nm, from about 700 nm to about 1200 nm, from about 800 nm to about 1200 nm, from about 900 nm to about 1200 nm, or from about 900 nm to about 1100 nm.

In some embodiments, the sensor composition of the present invention further comprises a stabilizer. In one embodiment, the sensor composition comprises a stabilizer comprising polyvinyl pyrrolidone (PVP).

In certain other embodiment, the sensor composition comprises a weight ratio of polymeric microparticles to stabilizer that is from about 1.5:1 to about 25:1, from about 1.5:1 to about 20:1, from about 2:1 to about 19:1, from about 2:1 to about 18:1, from about 2:1 to about 17:1, from about 2:1 to about 16:1, from about 2:1 to about 15:1, from about 2:1 to about 14:1, from about 2:1 to about 13:1, from about 2:1 to about 12:1, from about 2:1 to about 11:1, or from about 2:1 to about 10:1. In certain embodiments, the sensor composition comprises a weight ratio of polymeric microparticles to stabilizer that is from about 2:1 to about 10:1.

In one embodiment, the dispersion medium comprises an alcohol. In another embodiment, the dispersion medium comprises an alcohol or water. In yet another embodiment, the dispersion medium comprises ethanol. In still a further embodiment, the dispersion medium comprises ethanol and water. In one embodiment, the dispersion medium comprises an alcohol or ethanol and water. In yet further embodiments, the dispersion medium comprises polyvinyl pyrrolidone. In one embodiment, the dispersion medium comprises polyvinyl pyrrolidone suspended in 50% ethanol (v/v in water).

Another aspect of the present invention is directed to a process for preparing a sensor composition. The process comprises mixing polymeric microparticles, an oxygen, humidity, carbon dioxide, temperature, or pH-responsive dye comprising a luminophore, and a solvent to form a mixture comprising dye-loaded polymeric microparticles comprising the oxygen, humidity, carbon dioxide, temperature, or pH-responsive dye at least partially immobilized thereon. The dye-loaded polymeric microparticles are separated from at least a portion of the solvent in the mixture. The dye-loaded polymeric microparticles are then dispersed in a dispersion medium to form the sensor composition.

Oxygen Sensor Patch

A further aspect of the present invention is directed to an oxygen sensor patch. The oxygen sensor patch comprises a substrate and the sensor composition, as described here, disposed thereon.

For example, in one embodiment, the oxygen sensor patch comprises a substrate, and a sensor composition disposed on the substrate, wherein the sensor composition comprises polymeric microparticles; an oxygen-responsive dye comprising a luminophore that is at least partially immobilized on the polymeric microparticles; and a dispersion medium comprising the polymeric microparticles dispersed therein. In another embodiment, the oxygen sensor patch comprises a substrate and a sensor composition disposed on the substrate, wherein the sensor composition comprises dye-loaded polymeric microparticles comprising an oxygen-responsive dye at least partially immobilized on the polymeric microparticles.

The substrate is generally selected based the oxygen permeability of the substrate material. For example, in one embodiment, the substrate is a polymer sheet or film having low oxygen permeability. In one embodiment, the substrate is a flexible substrate. In a further embodiment, the substrate is a transparent substrate. In another embodiment, the substrate is a flexible substrate comprising polyvinylidene chloride (PVDC) and/or equivalent materials with low oxygen permeability.

In certain embodiments, the substrate may be at least partially coated. In another embodiment, the substrate may comprise a material having a high oxygen permeability. For example, in certain embodiments, the substrate is at least partially coated with a layer of polyethylene (PE), polydimethylsiloxane (PDMS), silicone, and/or equivalent materials with high oxygen permeability.

In one embodiment, the oxygen sensor patch comprises one or more additional protective layers or coatings on the one or more surfaces of the oxygen sensor patch. For example, the one or more additional protective layers or coatings may be selected based upon their ability to provide mechanical protection of the oxygen sensor patch. In another example, the one or more additional protective layers or coatings may be selected based upon their ability to provide chemical protection of the oxygen sensor patch.

A further aspect of the present invention is directed to a process for preparing an oxygen sensor patch as described herein. The process comprises applying the sensor composition as described herein to a surface of the substrate and drying the substrate to at least partially remove the dispersion medium.

The oxygen sensor patch of the present invention may be prepared by applying the sensor composition in any known manner of application. For example, in one embodiment, a single dot oxygen sensor patch is prepared by manual deposition of a certain amount of the sensor composition (e.g., 10 μL) on the surface of a substrate (e.g., PVDC). In another embodiment, an oxygen sensor patch comprises an array of sensor dots using an automated micro-dispensing system. The micro-dispensing system may be any micro-dispensing system suitable for deposition of the sensor composition.

Wherein the oxygen sensor patch comprises an array of sensor dots, the sensor dots may be derived from the same or different sensor compositions and may be on the same or different side of the oxygen sensor patch. For example, in one embodiment, the sensor patch comprises one or more “calibration sensor dot” exposed to an external environment and one or more “working sensor dot” exposed to the target material. For example, the one or more calibration sensor dot may be exposed to an external environment such as ambient air and the one or more working sensor dot may be exposed to biological tissue. In this embodiment, it is possible to directly compare the oxygen sensor dot data for the calibration sensor dot exposed to 21% oxygen (i.e. ambient air) and the working sensor dot exposed to the biological tissue. In certain other embodiments, the oxygen sensor patch comprises an array of calibration sensor dots, wherein the one or more calibration sensor dots are the same or different and exposed to the same or different conditions (e.g., a low-point calibration sensor and a high-point calibration sensor). In one embodiment, the oxygen sensor patch comprises an array of sensors dots including a working sensor dot, low-calibration sensor dot (for calibrating exposure to 0% oxygen) and high-point calibration sensor dot (for calibrating exposure to 21% oxygen). The use of multiple sensor dots provides more points of measurement along the surface of the sample, thereby increasing the accuracy and offsetting error attributable to the sensing of an individual dot. Further, the use of multiple sensor dots minimizes the inherent biologic variability of the oxygen level at different points in a biological tissue sample. For example, variability of the oxygen level as seen around a high pressure point, such as tissue over a bone.

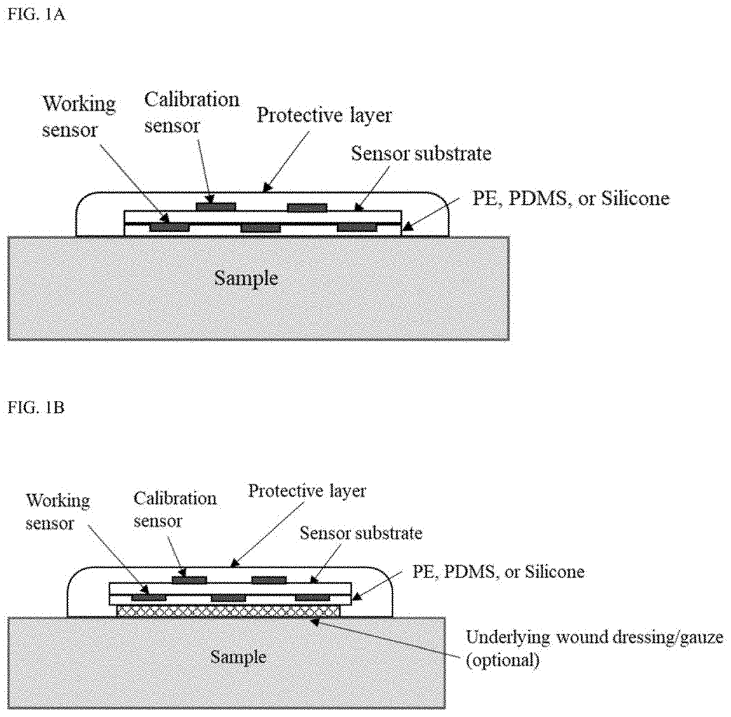

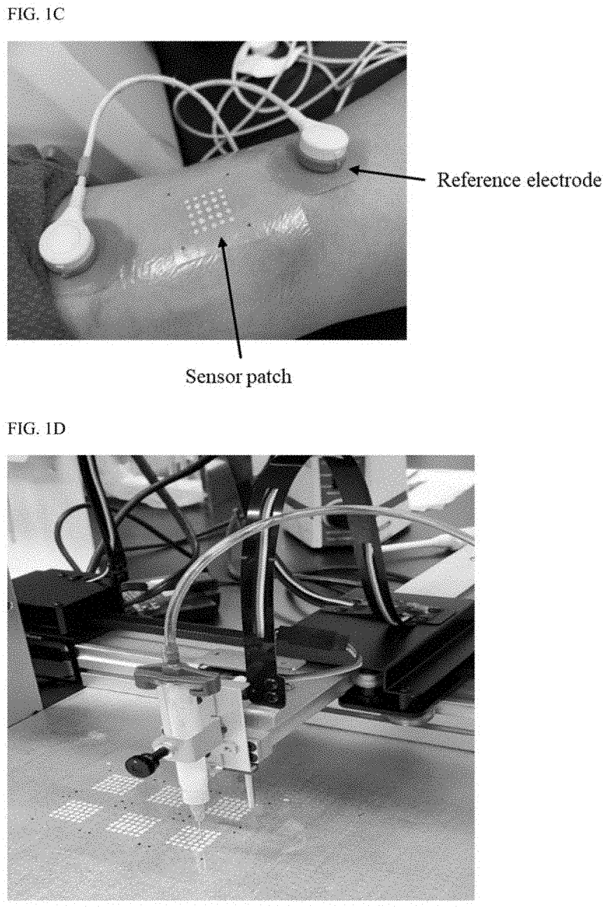

An exemplary configuration of a sensor patch comprising an array of sensor dots can be seen in FIG. 1A.

Still further aspects of the present invention are directed to methods of using an oxygen sensor patch as described herein.

In one embodiment, a method of monitoring a biological tissue is provided. The method comprises applying the oxygen sensor patch to at least a portion of a biological tissue and analyzing the luminescence of the sensor patch provides information about, for example, the transcutaneous oxygen tension of the biological tissue.

Humidity, Carbon Dioxide, Temperature, or pH Sensor Patch

Likewise, a further aspect of the present invention is directed to a humidity, carbon dioxide, temperature, or pH sensor patch. The humidity, carbon dioxide, temperature, or pH sensor patch comprises a substrate and the sensor composition, as described here, disposed thereon.

For example, in one embodiment, the humidity, carbon dioxide, temperature, or pH sensor patch comprises a substrate, and a sensor composition disposed on the substrate, wherein the sensor composition comprises polymeric microparticles; a humidity, carbon dioxide, temperature, or pH-responsive dye comprising a luminophore that is at least partially immobilized on the polymeric microparticles; and a dispersion medium comprising the polymeric microparticles dispersed therein. In another embodiment, the humidity, carbon dioxide, temperature, or pH sensor patch comprises a substrate and a sensor composition disposed on the substrate, wherein the sensor composition comprises dye-loaded polymeric microparticles comprising a humidity, carbon dioxide, temperature, or pH-responsive dye at least partially immobilized on the polymeric microparticles.

The substrate is generally selected based the permeability of the substrate material. In one embodiment, the substrate is a polymer sheet or film. In another embodiment, the substrate is a flexible substrate. In a further embodiment, the substrate is a transparent substrate. In still a further embodiment, the substrate is a flexible substrate comprising polyvinylidene chloride (PVDC) and/or equivalent materials.

In certain embodiments, the substrate may be at least partially coated. For example, in certain embodiments, the substrate is at least partially coated with a layer of polyethylene (PE), polydimethylsiloxane (PDMS), silicone, and/or equivalent materials.

In one embodiment, the humidity, carbon dioxide, temperature, or pH sensor patch comprises one or more additional protective layers or coatings on the one or more surfaces of the humidity, carbon dioxide, temperature, or pH sensor patch. For example, the one or more additional protective layers or coatings may be selected based upon their ability to provide mechanical protection of the humidity, carbon dioxide, temperature, or pH sensor patch. In another example, the one or more additional protective layers or coatings may be selected based upon their ability to provide chemical protection of the humidity, carbon dioxide, temperature, or pH sensor patch.

A further aspect of the present invention is directed to a process for preparing a humidity, carbon dioxide, temperature, or pH sensor patch as described herein. The process comprises applying the sensor composition as described herein to a surface of the substrate and drying the substrate to at least partially remove the dispersion medium.

The humidity, carbon dioxide, temperature, or pH sensor patch of the present invention may be prepared by applying the sensor composition in any known manner of application. For example, in one embodiment, a single dot humidity, carbon dioxide, temperature, or pH sensor patch is prepared by manual deposition of a certain amount of the sensor composition (e.g., 10 μL) on the surface of a substrate (e.g., PVDC). In another embodiment, a humidity, carbon dioxide, temperature, or pH sensor patch comprising an array of sensor dots using an automated micro-dispensing system. The micro-dispensing system may be any micro-dispensing system suitable for deposition of the sensor composition.

Wherein the humidity, carbon dioxide, temperature, or pH sensor patch comprises an array of sensor dots, the sensor dots may be derived from the same or different sensor compositions and may be on the same or different side of the sensor patch. For example, in one embodiment, the sensor patch comprises one or more “calibration sensor dot” exposed to an external environment and one or more “working sensor dot” exposed to the target material. For example, the one or more calibration sensor dot may be exposed to an external environment such as ambient air and the one or more working sensor dot may be exposed to biological tissue. In this embodiment, it is possible to directly compare the sensor dot data for the calibration sensor dot exposed to an external environment and the working sensor dot exposed to the biological tissue. In certain other embodiments, the sensor patch comprises an array of calibration sensor dots, wherein the one or more calibration sensor dots are the same or different and exposed to the same or different conditions (e.g., a low-point calibration sensor and a high-point calibration sensor). In one embodiment, the sensor patch comprises an array of sensors dots including a working sensor dot, low-calibration sensor dot, and high-point calibration sensor dot.

In certain embodiments, the oxygen, humidity, carbon dioxide, temperature, or pH sensor patch may be applied to a biological tissue that comprises one or more of an ulcer or wound. In another embodiment, the biological tissue may comprise a potential ulcer or wound or a skin area prone to develop an ulcer or wound. In another embodiment, the biological tissue may comprise any defect in the epidermis such that the dermis is visible. The biological tissue may also be any point over a bony surface (e.g., such as coccyx or calcaneus) that is subject to pressure (e.g., from a bed or other surface). The biological tissue may also be any area on a foot. The biological tissue of interest may particularly be the foot, when the subject is suffering from diabetes or is subject to increased pressure in ordinary daily activities, such as walking.

In certain embodiments, the oxygen, humidity, carbon dioxide, temperature, or pH sensor patch may be applied to a non-biological material selected from the group consisting of a solid material, a soft material, or an opening on a solid container or soft container.

In still a further embodiment, the sensor patch comprising an array of sensor dots may be applied to the surface of a sample (e.g., a biological tissues) wherein an underlying wound dressing layer is present between at least a portion of the surface of the sample and the sensor patch. An exemplary illustration of such a configuration is set forth in FIG. 1B. The underlying wound dressing layer may comprise any suitable wound dressing, for example, selected from the group consisting of gauze, lint, plaster, cotton, a bandage, and combinations thereof.

The analysis of the luminescence of the sensor patch may be conducted via any conventional means. For example, the luminescence may be analyzed with a multi frequency phase fluorometer (e.g., MFPF 100, Tau Theta Instruments/Ocean Optics, Inc. Dunedin, Fla., USA) or equivalent instruments.

The analysis of the luminescence of the sensor patch may generally comprise illuminating the sensor patch; magnifying the illumination; and capturing an image of the illuminated patch.

The analysis may be conducted via a computing device. In certain embodiments, the analysis may be conducted via a mobile computing device. In one embodiment, the mobile computing device is a consumer grade smartphone. In this embodiment, the mobile computing device comprises a plurality of LEDs and a camera comprising at least about a 10× macro lens assembly, internally or externally affixed. For example, the plurality of LEDs and a camera comprising at least about a 10× macro lens assembly can be externally affixed to the mobile computing device. The LEDs may be selected such that they have a peak wavelength in the range of from about 300 nm to about 500 nm, from about 310 nm to about 490 nm, from about 320 nm to about 480 nm, from about 330 nm to about 470 nm, from about 340 nm to about 460 nm, from about 350 nm to about 450 nm, from about 360 nm to about 440 nm, from about 370 nm to about 430 nm, from about 380 nm to about 420 nm, or from about 390 nm to about 420 nm. In certain embodiments, the LEDs have a have a peak wavelength in the range of from about 390 nm to about 420 nm.

Further processing of the results from analyzing the luminescence of the sensor patch may be conducted using a computer program comprising image analysis, calibration, calculation, and/or plotting and data transmission functions. For example, a diagnosis based on the luminescence of the sensor patch may be performed using a computer program comprising image analysis, calibration, calculation, and/or plotting and data transmission functions.

It was discovered that the phosphorescence intensity of the sensor composition of the present invention varied primarily with fluorophore concentration and to a lesser extent with polystyrene particle size. It was also discovered that the response of the sensor patch of the present invention was generally linear, stable and reproducible over an acceptable range of measurements. For example, it was discovered that the response of the oxygen sensor patch of the present invention was generally linear, stable and reproducible over a range of at least about 0 to about 21% oxygen. Support for these propositions can be seen in the examples set forth below.

### EXAMPLES

The following non-limiting examples are provided to further illustrate the present invention.

As used throughout the Examples, styrene monomer (St, 99%) 2,2′-azobis(2-methylpropionitrile) (AIBN, 98%) and two types of polyvinyl pyrrolidone (PVP) of molecular weight 360,000 and 40,000 are commercially available from Sigma-Aldrich (St. Louis, Mo., USA). Fluorophore Pt(II) meso-tetra (pentafluorophenyl)porphine (PtTFPP) is commercially available from Frontier Scientific (Logan, Utah, USA). Solvents like tetrahydrofuran (99.9%) and toluene (99.5%) are commercially available from Fischer Scientific (Pittsburgh, Pa., USA). Ethyl alcohol (99.5%) is commercially available from Acros Organics (Belgium, Wis., USA). Polyvinylidene chloride film (PVDC, thickness 0.033 mm) is commercially available from Goodfellow (Coraopolis, Pa., USA).

### Example 1: Preparation of Polymeric Microparticles

Polystyrene microparticles were prepared using the free radical dispersion polymerization technique. A dispersion medium was prepared in a 3-neck round bottom flask by dissolving 300 mg of PVP (MW 40,000, 5 wt % of styrene) in 12 mL of deionized water. 60 mg of AIBN (1 wt % of styrene) was dissolved in 6 g of purified styrene. The dissolved AIBN and 38 mL of ethanol were then added into the flask. The mixture was stirred at 70° C. for 24 h. After polymerization, a white suspension was formed. The suspension was centrifuged at 5000 rpm for 15 min, the supernatant was discarded, and the suspension was washed twice.

The particle size of the resulting microparticles were measured using a Zetasizer dynamic light scattering device (commercially available from Malvern Panalytical, Nano ZS90).

Additional polystyrene microparticles were also synthesized using 450 mg and 600 mg of PVP (7.5 and 10 wt % of styrene) respectively to evaluate the effect of the dispersion medium on the particle size. Solid microparticles were suspended in ethanol and particle concentration in the suspension was estimated after drying small portions under vacuum for 24 h.

### Example 2: Preparing a Sensor Composition

A suspension containing 100 mg of the polystyrene microparticles of Example 1 was centrifuged and re-suspended in 5 mL of 50% ethanol (v/v in water). 1 mg of a PtTFPP fluorophore was dissolved in 400 μL of THF. The dissolved PtTFPP was then added to the re-suspended polystyrene microparticles and the mixture was vortexed for 1 min to allow the absorption of the fluorophore on the microparticles. This diffusion and entrapment method allowed for the water-insoluble fluorophore to diffuse into the polymer matrix and become entrapped in the polystyrene microparticles.

The mixture was then centrifuged and the supernatant was discarded. The resulting fluorophore immobilized polystyrene microparticles were again suspended in 50% ethanol and fluorescence scans were performed using a Fluoroscence Spectroscope FS5 (commercially available from Edinburgh Instruments Ltd.).

A sensor composition was prepared by suspending the fluorophore immobilized polystyrene microparticles in a suitable dispersion medium i.e., 50% ethanol containing 1 wt % PVP (MW 360,000). In the sensor composition, PVP was used for two distinct purposes: as a dispersion medium in the liquid phase as well as adhesion promoter for the polystyrene microparticles after the sensor composition was applied to the sensor patch substrate and fluorophore dried on the substrate.

### Example 3: Average Particle Size Vs. Stabilizer (PVP) Ratio

To evaluate the effect of particle size on intensity, sensor compositions comprising polystyrene microparticles of differing particle size were prepared. 1 mg of fluorophore was immobilized on 100 mg of polystyrene microparticles of three different sizes and suspended in 3 mL of solution dispersion medium.

During dispersion polymerization, polystyrene particle size can be controlled by the amount of initiator, monomer, steric stabilizer, and polarity of the reaction media. In particular, the particle size was varied by choosing the steric stabilizer amount to alter the reaction condition. Polyvinyl pyrrolidone (PVP) proved to be a suitable steric stabilizer for dispersion polymerization.

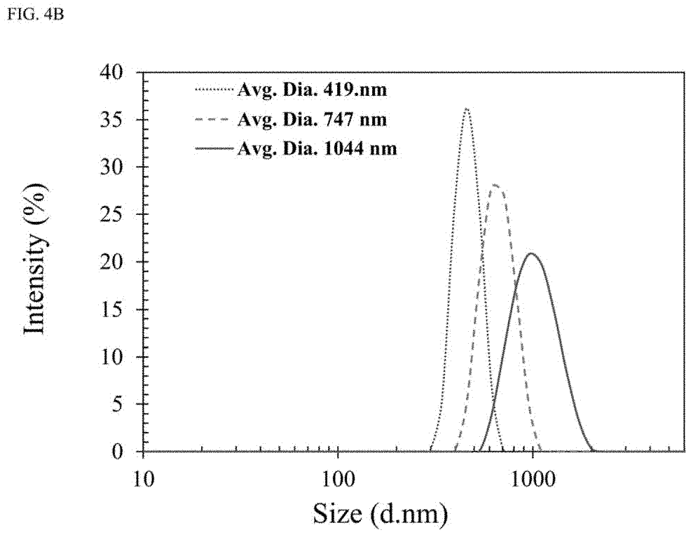

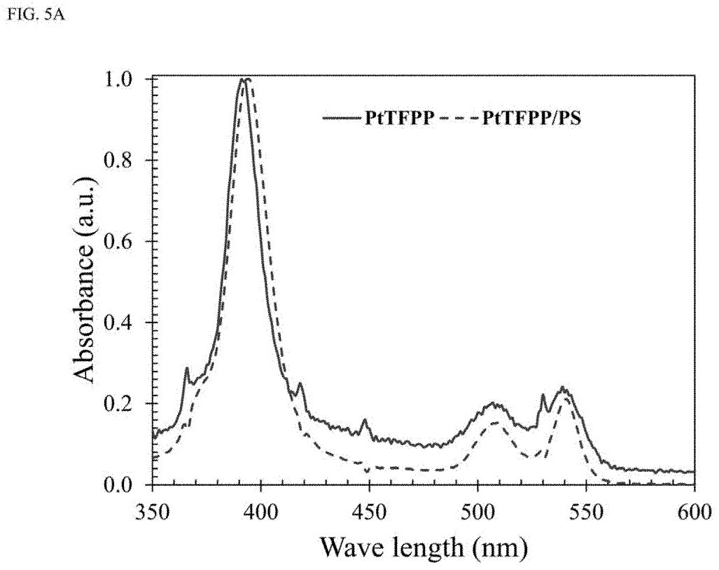

FIG. 4A shows the variation of polystyrene particle size with PVP-to-styrene-monomer ratio in the reaction vessel. Resultant polystyrene microparticles were of three different sizes ranging from approximately 0.4 to 1.0 μm. PVP is thought to stabilize the polystyrene latex by adsorbing on the particle surface. Thus, the average particle size gradually decreases as the PVP content is increased.

Size distribution of the three particle sizes is shown in FIG. 4B. This figure demonstrates that a narrower size distribution may result from a lower particle size.

### Example 4: Preparing an Oxygen Sensor Patch Fabrication Device

A single dot oxygen sensor patch was fabricated by manual deposition of 10 μL of a sensor composition on a PVDC film substrate and allowing it to dry in a dark place.

An oxygen sensor patch with an array of sensor dots (i.e., dots comprising the sensor composition) was also printed on a PVDC film substrate using an automated micro-dispensing system suitable for particle-based compositions.

A prototype printer was also built using a Nordson EFD (commercially available from Nordson Corporation, Westlake, Ohio, USA) automated droplet deposition system attached with an AxiDraw V3 2.5D Plotter (commercially available from Evil Mad Scientist Laboratories, Sunnyvale, Calif., USA) and a house-built platform, shown in FIG. 1D. The printer was a low-volume precision dispensing device operated by simple laboratory-scale pneumatic pressure. The size of the printed sensor dots was effectively controlled by dispensed volume of the sensor composition and by means of the needle gauge attached to the printing head as well as the pneumatic pressure utilized.

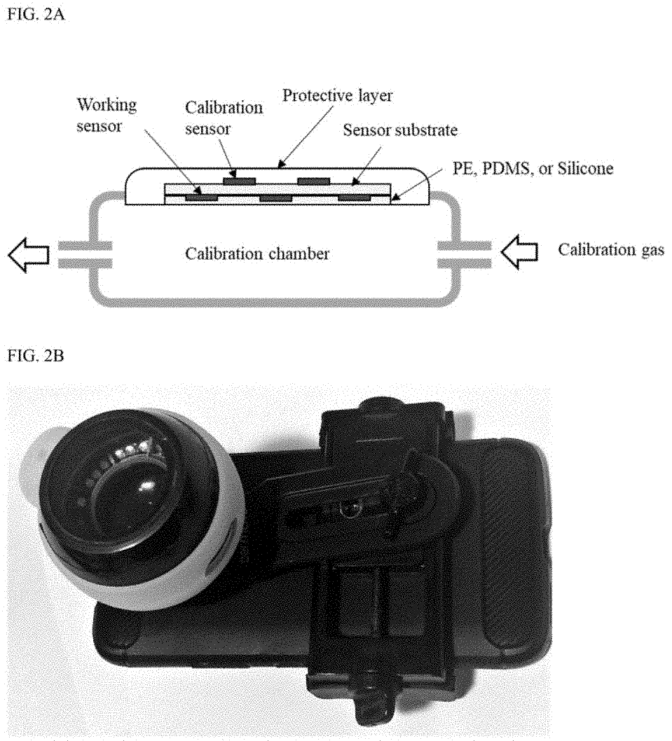

For potential self-calibration, five sensor dots were printed on the opposite side of the film (FIG. 2A) arranged in a quincunx pattern and always exposed to air (i.e., 21% oxygen) to serve as visual calibration sensors or calibration sensor dots. These calibration sensor dots are intended to be utilized for evaluating the intensity change over time for long-term applications. For example, by comparison of the working sensors to the calibration sensors. In one embodiment, the protective layer of FIG. 2A is oxygen permeable layer. The quincunx pattern is an example of a configuration that enables close proximity of all sensor dots to the calibration dots. However, other patterns could be employed.

### Example 5: Evaluating the Oxygen Sensor Patch

The luminescence response of the single-dot oxygen sensor patch of Example 4 was measured at different oxygen concentrations present in the gas phase by a multi-frequency phase fluorometer (MFPF 100, commercially available from Tau Theta Instruments/Ocean Optics, Inc. Dunedin, Fla., USA) affixed with a 470 nm LED for excitation.

An alternate image acquisition technique was also employed to determine the luminescence response from the oxygen sensor patch using a consumer-grade Android smart phone. The sensor patch was illuminated using twenty-four 405 nm LEDs arranged in a circular layout, magnified using a 10× macro lens assembly and passed through a high pass optical filter with a cut-on wavelength of 600 nm. The entire assembly was externally attached to the smart phone, as shown in FIG. 2B. The 405 nm LEDs were chosen based upon the 405 nm wavelengths ability to excite the maximum luminescence response from the oxygen sensor patch.

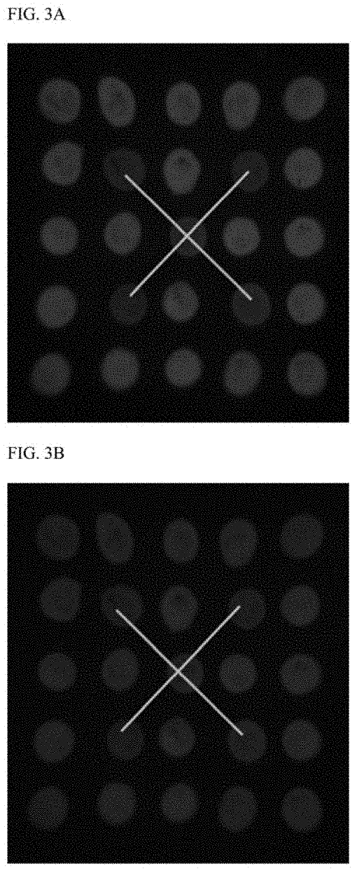

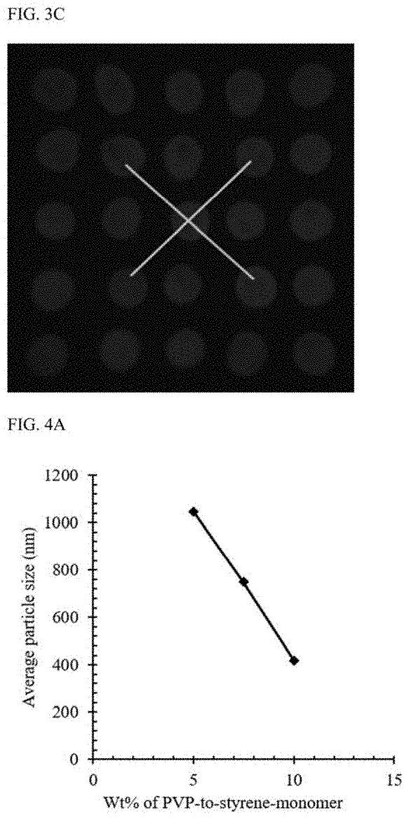

FIG. 3A through 3C show the luminescence response captured by the smart phone and saved in RAW format. FIG. 3A represented exposure to 0% oxygen. FIG. 3B represented exposure to 4% oxygen. FIG. 3C represented exposure to 12% oxygen. RAW is the industry standard format for serializing image sensor data. This represents data similar to the response produced by a silicon sensor. Typically, the sensor data is only processed based on a Bayer filter. A Bayer filter is a color filter array that used to decode sensor data to RGB image data. The images of this example were further processed using MATLAB® software, to obtain the luminescence sensor response.

For both the multi-frequency phase fluorometer and consumer-grade Android smart phone techniques, the oxygen sensor patches were attached to a calibration chamber as shown, for example, in FIG. 2A. Pure nitrogen and oxygen gases were directed to the calibration chamber as the calibration gas, and the respective flow rates were controlled by two mass flow controllers in order to achieve the desired oxygen concentration in the chamber. Phosphorescence intensity under different oxygen concentrations (0-21%) were measured and the Stern-Volmer plots were established.

### Example 6: Characterization of Dye-Loaded Polystyrene Microparticles

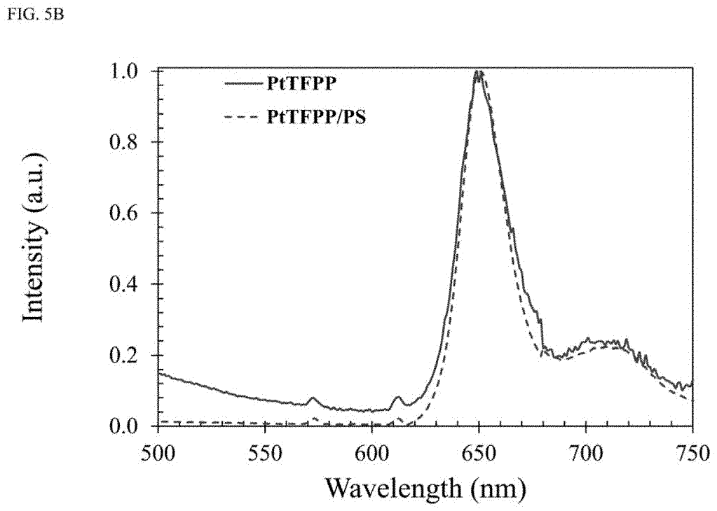

The dye diffusion and entrapment method allows homogenous distribution of fluorophore into the polystyrene microparticles without forming any covalent bond on its surface. FIGS. 5A and 5B show the normalized absorption and emission spectra of the PtTFPP dye and dye encapsulated in a polystyrene microparticle (hereinafter PtTFPP/PS) respectively suspended in 50% ethanol. To minimize the scattering effect from the suspended microparticles, cuvettes were placed at 45° inclined position in the cuvette holder of the Fluorescence Spectroscope FS5. Furthermore, spectra for the PtTFPP/PS particle suspension were plotted after subtracting a blank polystyrene microparticle spectra from them, to remove the effect of scattering. FIG. 5A shows that PtTFPP dye has a Soret band at 394 nm followed by two Q bands at 508 nm and 540 nm, respectively. PtTFPP has emission maxima at 650 nm, as seen in FIG. 5B. Absorption and emission maxima for PtTFPP/PS particles were similar to those of the PtTFPP dye alone, which signifies that adsorption of PtTFPP on the polystyrene microparticles surface did not alter the luminescence characteristics of the fluorophore.

### Example 7: Luminescence Intensity Vs. Polystyrene Particle Size and Dye Concentration

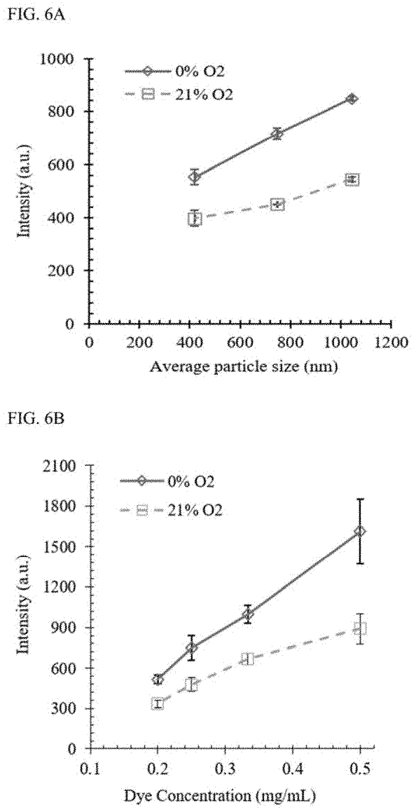

Three types of inks prepared with three different sizes of polystyrene microparticles were tested for particle size-luminescence intensity correlation. FIG. 6A represents the fluorescence intensity of the sensor patches measured with MFPF 100. This figure shows that the intensity increased with particle size in both 0% and 21% oxygen environments without altering the sensor performance. Such a result may be attributable to an increased cross section with larger particle size, which would lead to more fluorophore being excited. I₀/I₂₁ was used as the measure of sensor sensitivity, where I₀ and 121 represents the phosphorescence intensity of the sensors exposed to 0% and 21% oxygen respectively. It was found that sensitivity (I₀/I₂₁) of the sensors was not altered significantly with particle size, and that approximately 1 m sized microparticles emitted a maximum fluorescence intensity while maintaining overall homogeneity of the sensor patch.

To test the fluorophore concentration effect on the oxygen sensor patch to which the sensor composition as applied, several sensor compositions were prepared. 1 mg of fluorophore was immobilized on 100 mg of polystyrene microparticles (˜1 m size). The fluorophore immobilized polystyrene microparticles were then suspended in 5 ml, 4 ml, 3 ml and 2 ml of dispersion medium, respectively. This resulted in a fluorophore concentration ranging from 0.2 to 0.5 mg/mL in the sensor composition. The sensor composition was applied to the oxygen sensor patch and the patches were tested with MFPF 100.

FIG. 6B shows the intensity of those patches measured at 0% and 21% oxygen concentrations. Intensity increased linearly for the 21% oxygen concentration sample with sensor composition concentration up to 0.33 mg/mL and then appeared to increase at a slower rate for higher sensor composition concentrations. It was theorized that this might have been attributable to self-quenching of the fluorophore and higher sensor composition concentrations.

### Example 8: Performance of an Oxygen Sensors

A series of single sensor dot oxygen sensor patches and oxygen sensor patches were prepared in accordance with the above examples and excited with a 470 nm light and 405 nm light, respectively.

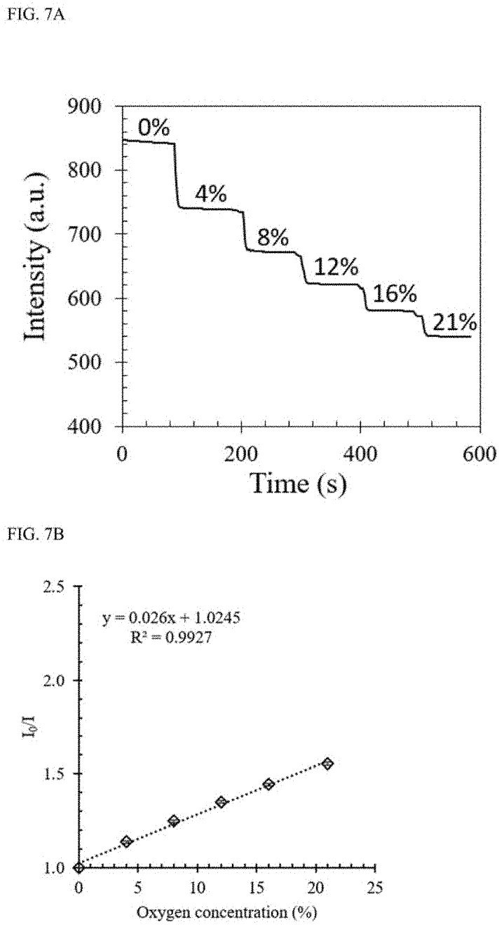

FIG. 7A shows the phosphorescence intensity emitted by the single sensor dot oxygen sensor patch when excited with 470 nm light at different oxygen concentration, measured with MFPF 100. Phosphorescence intensity decreased significantly with increase in oxygen concentration. FIG. 7B reports the Stern-Volmer plot established for the single sensor dot oxygen sensor patch when excited with 470 nm light.

FIG. 9 represents the operational stability of the single sensor dot oxygen sensor patch excited with 470 nm light. It was observed that the sensor response was very stable and reproducible when switching between a fully oxygenated to fully deoxygenated atmosphere. Response time and recovery time of the sensor were evaluated based on t₉₅ (i.e. the time required to achieve 95% of the total intensity change when switching between oxygen and nitrogen environments). Response time of the sensor to fully oxygenated condition was 4 seconds, whereas recovery time to fully deoxygenated condition was 20 seconds. The short response time makes it suitable for rapid detection of gaseous oxygen concentration in many practical applications.

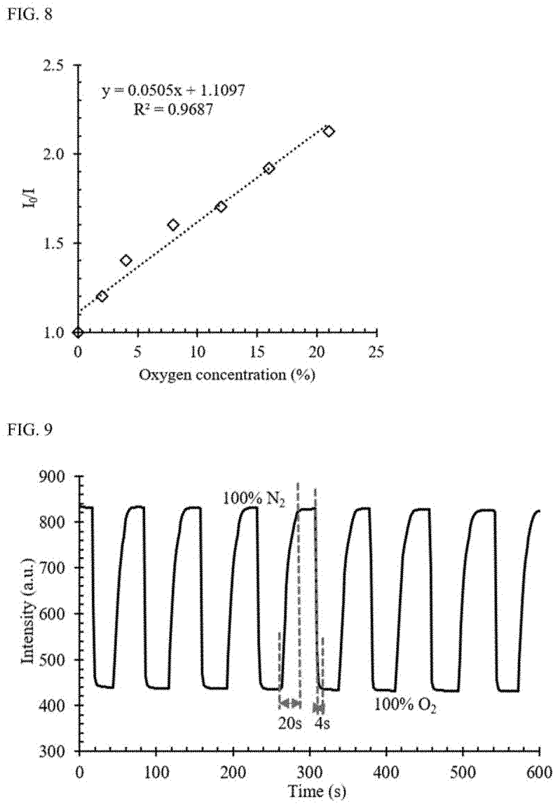

FIG. 8 shows the Stern-Volmer plot for an array of sensors on an oxygen sensor patch, excited with 405 nm light and measured for phosphorescence intensity using the smart phone techniques described above. Each data point represents the average of all working sensors in a respective image at the respective oxygen concentration.

In both of the Stern-Volmer plots, a linear response up to 21% oxygen was observed. This is considered to be suitable for most common clinical and environmental applications. Sensitivity between 0-21% obtained for the sensor array oxygen sensor patch was 2.13, which was higher than the sensitivity of single sensor dot oxygen sensor patch, i.e., 1.55. This lower sensitivity was expected since the single sensor dot oxygen sensor patch were excited with 470 nm light (the only wavelength available from MFPF), whereas the sensor array oxygen sensor patch was illuminated with 405 nm light. The illumination with 405 nm light is much closer to the Soret band of the PtTFPP fluorophore used in the examples. The sensor response was very fast and reproducible with a relative standard deviation of less than 1.5%.

The oxygen sensor patches of this example, along with the imaging processing technique, made the system suitable for simple, cost-effective monitoring of oxygen on a surface. Wide dynamic linear range (0-21% oxygen) of the sensors covers the physiological range. Therefore, it is expected that the oxygen sensor patch is an ideal platform for early detection and timely intervention of surface wounds associated with tissue oxygen.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above sensor products, compositions, methods of construction, and methods of use, without departing from the scope of the invention, it is intended that all matter contained in the above description and shown in the accompanying drawings shall be interpreted as illustrative and not in a limiting sense.

Having described the invention in detail, it will be apparent that modifications and variations are possible without departing from the scope of the invention defined in the appended claims.

1. A sensor composition comprising:
polymeric microparticles; a dye comprising a luminophore that is at least partially immobilized on the polymeric microparticles; and a dispersion medium comprising the polymeric microparticles dispersed therein.

2. A process for preparing a sensor composition comprising:
mixing polymeric microparticles, a dye comprising a luminophore, and a solvent to form a mixture comprising dye-loaded polymeric microparticles comprising the dye at least partially immobilized thereon; separating the dye-loaded polymeric microparticles from at least a portion of the solvent in the mixture; and dispersing the dye-loaded polymeric microparticles in a dispersion medium to form the sensor composition.

3. The composition of claim 1, wherein the luminophore comprises a fluorophore or a phosphor that is responsive to one or more of oxygen, humidity, carbon dioxide, temperature, or pH.

4. The process of claim 2, wherein the luminophore comprises a fluorophore or a phosphor that is responsive to one or more of oxygen, humidity, carbon dioxide, temperature, or pH.

5. The composition of claim 3, wherein the fluorophore is selected from the group consisting of platinum(II) meso-tetra(pentafluorophenyl)porphin (PtTFPP), platinum(II) meso-tetraphenyl tetrabenzoporphine (PtTPTBP), and combinations thereof.

6. The composition of claim 1, wherein the luminophore has a peak emission wavelength from about 300 nm to about 800 nm, from about 300 nm to about 700 nm, from about 300 nm to about 600 nm, from about 300 nm to about 500 nm, from about 310 nm to about 490 nm, from about 320 nm to about 480 nm, from about 330 nm to about 470 nm, from about 340 nm to about 460 nm, from about 350 nm to about 450 nm, from about 360 nm to about 440 nm, from about 370 nm to about 430 nm, from about 380 nm to about 420 nm, or from about 390 nm to about 410 nm.

7. The composition of claim 1, wherein the polymeric microparticles comprise polystyrene microparticles having an average particle size of from about 300 nm to about 1200 nm, from about 400 nm to about 1200 nm, from about 500 nm to about 1200 nm, from about 600 nm to about 1200 nm, from about 700 nm to about 1200 nm, from about 800 nm to about 1200 nm, from about 900 nm to about 1200 nm, or from about 900 nm to about 1100 nm.

8. The composition of claim 1, wherein the composition further comprises a stabilizer and has weight ratio of polymeric microparticles to stabilizer that is from about 1.5:1 to about 25:1, from about 1.5:1 to about 20:1, from about 2:1 to about 19:1, from about 2:1 to about 18:1, from about 2:1 to about 17:1, from about 2:1 to about 16:1, from about 2:1 to about 15:1, from about 2:1 to about 14:1, from about 2:1 to about 13:1, from about 2:1 to about 12:1, from about 2:1 to about 11:1, or from about 2:1 to about 10:1.

9. A sensor patch comprising:
a substrate having
(a) the sensor composition of claim 1 disposed thereon; or
(b) a sensor composition disposed thereon, wherein the sensor composition comprises dye-loaded polymeric microparticles comprising an oxygen, humidity, carbon dioxide, temperature, or pH-responsive dye at least partially immobilized on polymeric microparticles.

10. The sensor patch of claim 9, wherein the substrate comprises polyvinylidene chloride (PVDC) and/or equivalent materials.

11. The sensor patch of claim 9, wherein the substrate is at least partially coated or laminated with a layer of polyethylene (PE), polydimethylsiloxane (PDMS), silicone, and/or equivalent materials.

12. The sensor patch of claim 9, wherein the sensor patch comprises one or more working sensor and one or more calibration sensor, and wherein the working sensor and calibration sensor are present on opposite sides of the sensor patch.

13. A process for preparing a sensor patch comprising:
applying the sensor composition of claim 1 to a surface of a substrate; and
drying the substrate to at least partially remove the dispersion medium.

14. A method of monitoring a sample comprising:
applying the sensor patch of claim 9 to the sample; and
analyzing the luminescence of the sensor patch.

15. The method of claim 14, wherein the sample comprises a biological tissue and wherein an underlying wound dressing material is present between at least a portion of the sensor patch and the biological tissue.

16. The method of claim 14, wherein analyzing the luminescence of the sensor patch comprises:
illuminating the sensor patch; magnifying the illumination; and capturing an image of the illuminated sensor patch.

17. The method of claim 16, wherein the analysis is conducted using a mobile computing device comprising a plurality of LEDs and a camera comprising at least about a 10× macro lens assembly, internally or externally affixed.

18. The method of claim 16, wherein the sensor patch comprises one or more working sensor and one or more calibration sensor, and wherein the working sensor and calibration sensor are present on opposite sides of the sensor patch; wherein the luminescence intensity of the one or more calibration sensor and one or more working sensor are recorded in the captured image; and the luminescence intensity of the calibration and working sensors are compared to calculate the oxygen value.

19. The method of claim 18, wherein the calibration sensors and working sensors are arranged in a quincunx pattern.

20. The method of claim 14, wherein the sample comprises one or more location of interest selected from the group consisting of:
a biological tissue selected from the group consisting of an ulcer, wound, a potential ulcer, a skin area prone to develop an ulcer or wound, any point over a bony surface, and any point subject to pressure;
a non-biological material selected from the group consisting of a solid material, a soft material, or an opening on a solid container or soft container;
and combination thereof.
